(19) United States
(12) Patent Application Publication
(10) Pub. No.: US 2016/0075478 A1
(43) Pub. Date: Mar. 17, 2016

(54) SOLVENT-ACTIVATED FILM FOR CONTAINER

(71) Applicants: Jameson Henry Godsey, Los Gatos, CA (US); Thomas Joshua Godsey, Los Gatos, CA (US); James Henry Godsey, Los Gatos, CA (US); Sandra Lynn Godsey, Los Gatos, CA (US)

(72) Inventors: Jameson Henry Godsey, Los Gatos, CA (US); Thomas Joshua Godsey, Los Gatos, CA (US); James Henry Godsey, Los Gatos, CA (US); Sandra Lynn Godsey, Los Gatos, CA (US)

(21) Appl. No.: 14/488,243

(22) Filed: Sep. 16, 2014

(51) Int. Cl. B65D 25/14; A61K 8/02; C09D 103/00; C11D 17/00; A61Q 11/00; A23L 2/395; A61K 9/70

(52) U.S. Cl. CPC B65D 25/14; A23L 2/395; A61K 8/0216; A61K 9/7007; A61K 8/0233; C11D 17/0095; A61Q 11/00; C09D 103/00; A23V 2002/00; A61K 2800/30

(57) ABSTRACT

A container having a solvent-activated film, wherein the solvent reconstitutes a content material in the film. In one embodiment, the film is a concentrated mouthwash which reacts when water is added to the container, such as a disposable cup, to reconstitute the mouthwash at a desired strength.

### FIELD OF THE INVENTION

The present invention relates to containers lined with material for consumption or use.

### BACKGROUND

Currently there are a variety of situations where a large volume of liquid is purchased and stored in home and business for applications such as mouth care, etc. For example, for mouthwash, the user purchase a bottle of liquid, adding to the volume and mass of the grocery purchase, weight in transport and storage within the home. The bottle is then opened and poured into a cup, often a disposable cup for use. A film often develops on the bottle, making it seem unclean and unsanitary.

### BRIEF DESCRIPTION OF THE DRAWINGS

FIG. 1 illustrates a system including a disposable paper cup, and a bottle of mouthwash, according to the prior art.

FIG. 2 illustrates a cup shape, according to the prior art.

FIG. 3 illustrates a film made up of a substance to be reconstituted in presence of a solvent, according to embodiments of the present invention.

FIG. 4 illustrates a cup structure having a film, according to embodiments of the present invention.

FIG. 5 illustrates a container cover structure having a film, according to embodiments of the present invention.

FIG. 6 illustrates a solvent-activated film deposited on a container material during the manufacturing of the container, according to example embodiments.

FIG. 7 illustrates a bottle according including a film that contains a concentrated content, according to embodiments of the present invention.

FIG. 8 illustrates a film structure, according to embodiments of the present invention.

FIG. 9 illustrates a container having a film on the inner surface, the film having multiple portions, according to embodiments of the present invention.

### DETAILED DESCRIPTION

The present invention as illustrated in examples and considered in a variety of applications is an apparatus and method for providing a solid component for a liquid as a liner within a container; wherein the liner is reconstituted with water at time of preparation for use. In some embodiments, a disposable cup includes a mouthwash layer which adheres to at least a portion of the inner surfaces of the disposable cup, wherein the mouthwash layer dissolves when a liquid, such as water, is added; the mouthwash layer dissolves to form liquid mouthwash. The mouthwash layer could be a material similar to a thin-film drug delivery system, such as breath strips or cold strips, which dissolve in the user's mouth. In this application, the film or layer dissolves when a liquid is introduced, and thus reconstitutes the desired liquid material.

In general, the container may be a one-time use or reusable container, wherein after the first use there may remain sufficient material to be reconstituted a second or multiple times. Similarly, the container may be reusable for other purposes, or may be a disposable container. The component may be a material that is provided as a formulation which allows a stable solid form that reacts with addition of another material. In some embodiments the additive is water, while in other embodiments the additive is a gas or solid formulation.

The component may be an oral drug strip type component which uses strip-forming polymers, plasticizers, gelatin, active pharmaceutical ingredient, sweetening agents, stimulating agents, flavoring agents, coloring agents, breath cleansing agents, stabilizing and thickening agents. In some embodiments the component may be exposed to air without significant degradation over a reasonable time period to allow manufacture, shipping, purchasing, and storing for later use.

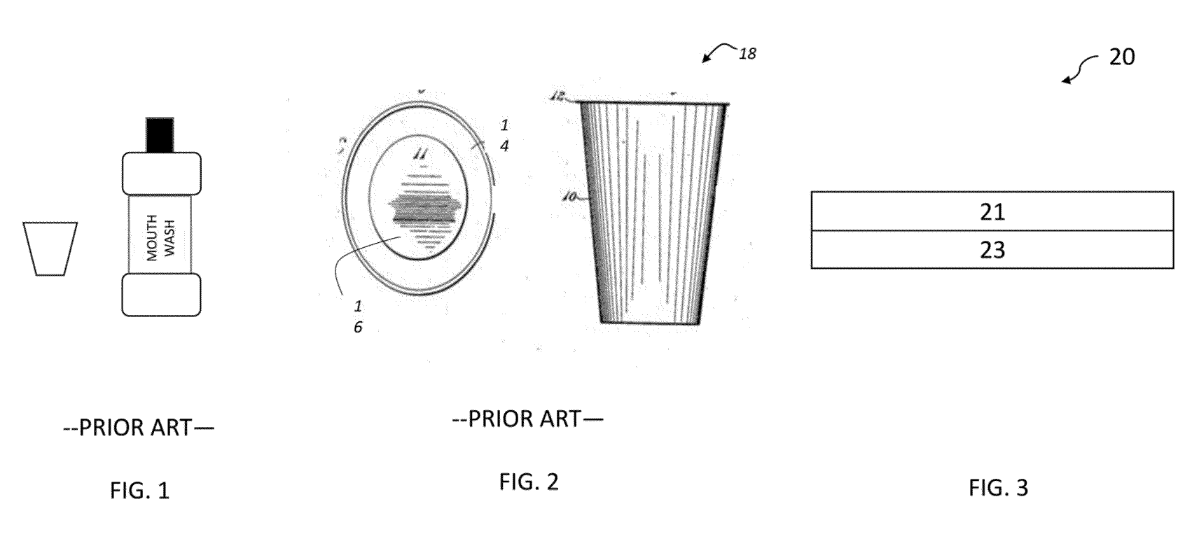

As illustrated in FIG. 1, the prior art system includes a disposable paper cup, and a bottle of mouthwash. This system requires multiple components, including a large bottle of liquid mouth wash or mouth rinse. In this system, the illustrated mouth wash is provided by Listerine®, by Johnson & Johnson Inc.

The mouth wash of FIG. 1 may be also be used with glass, other cups or containers, wherein the mouth wash may be used full strength or may be diluted with water. In some embodiments, the present invention enables the user to add the water required to make the mouth wash their desired strength, similar to the current use by the user, where water is added to the mouth wash liquid from the bottle.

The prior art container 18 illustrated in FIG. 2, has a cup shape with a top rim 12 connected to a wall structure having an inner surface 14 and an outer surface 10. The container 18 also has a bottom portion 16 connected to the wall structure. In the container 18 of FIG. 2, the inner surface 14 is a material allowing liquid to be placed in the container 18 for a given duration, which may be a few hours, such as a paper cup, or may be indefinite, such as a plastic cup.

An embodiment of the present invention is illustrated, as applied to the container 18 of FIG. 2, wherein the container's inner surface has a film or coating made up of a substance to be reconstituted or dissolved in presence of a solvent, and is illustrated in FIG. 3. The film 20 includes a content layer 21 and an adhesive layer 23. The content layer 21 contains the active ingredient, such as mouthwash or medicine. The content layer 21 is made of an edible film provided as a thin layer of consumable material. The film provides a barrier to environmental conditions, such as moisture, temperature and pressure. The adhesive layer 23 acts to bind the content layer 21 to a surface of a container, such as a cup or bottle. The adhesive layer 23 is made of an edible material that either dissolves when the content layer 21 is reconstituted, or may persist on the surface to which it is attached. According to some embodiments, the adhesive layer 23 and the content layer 21 are combined into a single film incorporating agents that ensure the stability of the structure as well as allow the reconstitution of the content when water or other solvent is added to the container.

The film 20 may comprise polysaccharides, including cellulose, starch derivatives, pectin derivatives, seaweed extracts, exudate gums, microbial fermentation gums and chitosan, and other materials. Polysaccharides tend to be hydrophilic resulting in poor water vapor and gas barrier properties, but enabling easy reconstitution of the active ingredients of the content layer 24.

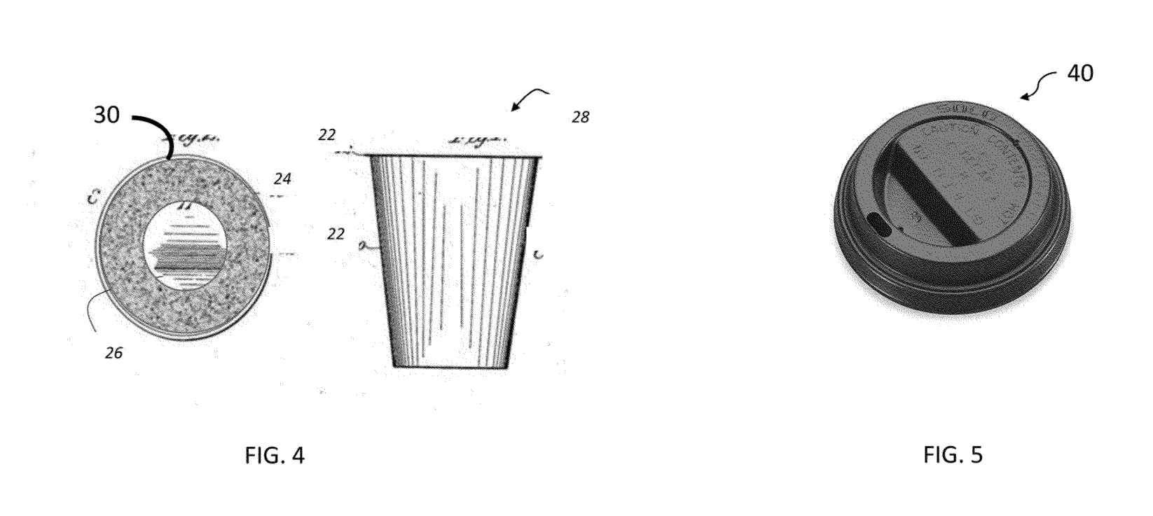

In some embodiments the substance is a film that is activated by water to form the full strength content, such as mouthwash. The film is then a concentrated form of the mouthwash which is provided in a solid (or semi-solid) form so as to adhere to the container. As illustrated in FIG. 4, a film 30 is positioned on the inner wall surface area 24 of the cup 28. In this way, water introduced to the cup 28 interacts with the film 30 so as to reconstitute the substance of the film 30 to full strength mouth wash. The film 30 dissolves when the solvent, water, is added. The cup 28 with the film 30 provides a user with a simple way to maintain proper oral health, and avoids the need for large bottles of liquid mouthwash. In some embodiments the film 30 may be formed or deposited on the inner bottom surface 26, or on a combination of the inner surfaces. In some embodiments the film may be provided as an oral strip, which is positioned within the cup and is attached with an edible or potable adhesive material.

As used herein, reconstitute refers to any change of the active ingredient in the content material of the film into a different form, such as a change in the concentration of the content material or an active ingredient of the content material. In some applications the content material, or active ingredient in the content material, has a predetermined or specific concentration value in relation to a given solvent; thee predetermined concentration value corresponds to a first use of the content material, or active ingredient. On reconstitution in the presence of a solvent, the content material of the film returns to and the predetermined concentration. For a given application, there may be multiple solvents to which the film reacts, for example, a specific medication may reconstitute with water or some other liquid. For a given application, there may be multiple concentration levels achieved by changing the amount of solvent added to the container, such as where the content material continues from a concentrated material to less concentrated values as additional solvent is presented.

In some embodiments the film 30 may be formed or deposited in the top rim 22, wherein the user may fill the cup with water, or other solvent, and a portion of the film is received when the user drinks from the cup. In addition to mouthwash, such embodiments may include a flavoring for a beverage, such as to add flavor to water or to add salt to a margarita drink. Still other application may employ a vitamin or medicinal compound which is ingested on drinking from the cup. This provides a pleasant way to take medications, or ensure that daily vitamins are received.

A polymer employed may be designed to be a non-toxic, non-irritant and devoid of leachable impurities. It should have good wetting and spreadability property. The polymer should exhibit sufficient peel, shear and tensile strengths. The polymer should be readily available and should not be very expensive. Film obtained should be tough enough so that there won't be any damage while handling or during transportation. Combinations of materials are used to formulate oral strips wherein pullulan is one of the most widely used film former (used in Listerine PocketPak, Benadryl, etc.)

Another component may be a plasticizer which improves the flexibility of the film or strip and reduces the brittleness. Plasticizer improves the strip properties by reducing the glass transition temperature of the polymer. Glycerol, Propylene glycol, low molecular weight polyethylene glycols, phthalate derivatives like dimethyl, diethyl and dibutyl phthalate, Citrate derivatives such as tributyl, triethyl, acetyl citrate, triacetin and castor oil are some of the commonly used plasticizer excipients.

Active ingredient(s) may include breath control materials, medicine, or other ingredients to achieve the desired result. In each application the active ingredient is part of a film on a surface of the container that is activated on use. In addition to the active ingredient(s), the film may include sweetening, flavoring and/or coloring agents. An aspect of thin film technology is its taste and color. The sweet taste in formulation is used where the active ingredient(s), and other components, may have a disagreeable taste and is particularly useful for pediatric applications. Natural sweeteners as well as artificial sweeteners are used to improve the flavor, and may allow for individual selection of flavors by users. For example, the film may be provided in a variety of flavors, or the film may result in different flavors depending on the solvent used to activate the film. Pigments may be incorporated for coloring.

In addition, embodiments may employ stabilizing and thickening agents to improve viscosity and consistency of the film. The film provided in a container may be any of a variety of types. In some embodiments the film is an edible or potable film. The film is a solvent-activated film and may be provided as a liner within a cup.

In some embodiments the film includes a concentrated beverage which reacts with the introduction of a solvent, such as a coffee filter that includes a coffee film, which turns to coffee when water is put through the filter. A similar application is for a tea film in a tea bag structure. The filter or tea bag could be made of an edible or potable material which dissolves on the addition of hot water into a beverage.

In some embodiments the film may be a beverage such as dried milk product which may be reconstituted with water, and may be useful for camping or emergency survival kits. The film may take the place of powdered drinks which are added to water; by providing such drinks as a film that lines a cup, the need for extra powdered drink containers is removed.

In some embodiments the film is positioned on a lid for a cup, such as illustrated in FIG. 5. Here the film is applied to the under portion of the lid near to the opening, and such that as the user drinks from the cup, the film substance is ingested. The film could be applied to a straw, which is then activated as solvent is sucked through the straw. In this way, a vitamin may be added to the straw for easy consumption. The film may be added to a cereal straw, such as those provided by Kellogg's. The film, such as a vitamin film, is deposited on or adhered to the inner surface of the straw, wherein the film reacts with milk as the user drinks through the cereal straw; here the solvent is milk.

In still other embodiments, the film is a concentrated water-based paint, which is then reacts with water to form liquid paint. This application reduces the heavy lifting associated with the purchase of gallons of paint. When the user desires to paint, they add water or other solvent to the container, and the film turns into the desired paint. Some embodiments may provide custom or specific colors of paint by using multiple different films, such as to form orange using shade(s) of yellow with shade(s) of red.

Further, household cleaners and detergents may be provided in film form, which are then r at time of use by addition of water or other solvent.

For the mouthwash application, the container may be any of a variety of materials. One material used for the disposable cup is paper or other renewable resource, which may be discarded or composted after use.

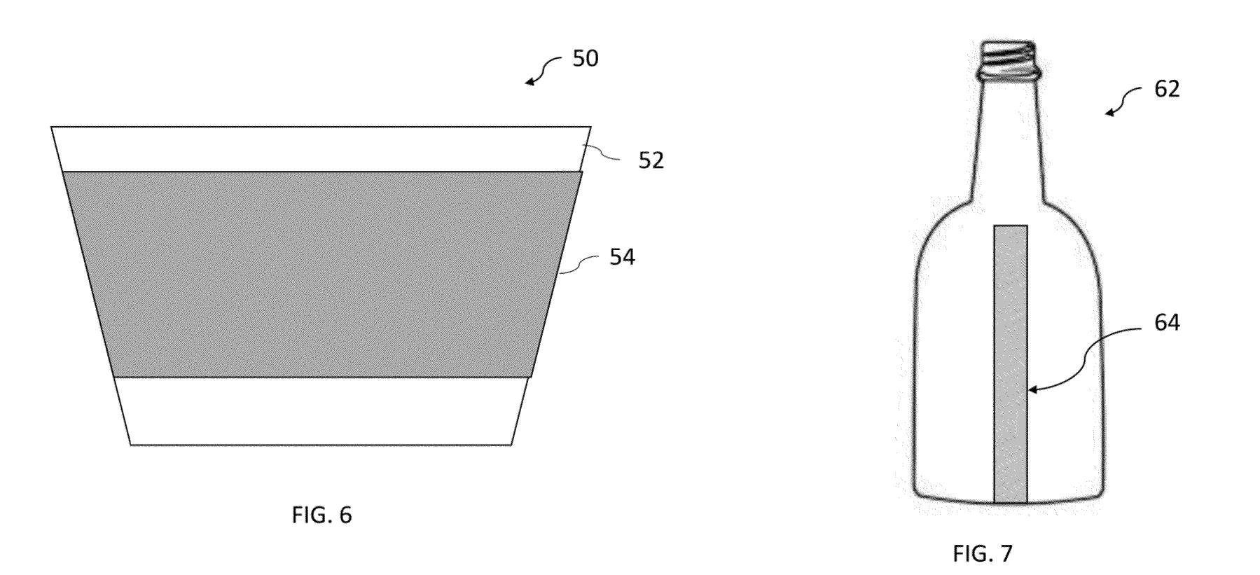

According to some embodiments, a solvent-activated film is deposited on container material during the manufacturing of the container. As illustrated in FIG. 6, wherein the container 50 is made of a precut or otherwise formed material 52. Prior to completing the form for the container 50, the film 54 is deposited, sprayed or dipped onto the inner surface of the material 52. The container formation is then completed.

There are a variety of materials that could be used for the film 54 such as a cellulose material, which may be configured as a tight packing of polymer chains and a highly crystalline structure that resists salvation in aqueous media. Water solubility may be increased by reacting the cellulose with chloroacetic acid, methyl chloride or propylene oxide to yield carboxymethyl cellulose (CMC), methyl cellulose (MC), hydroxypropyl cellulose (HPMC) or hydroxypropyl cellulose (HPC).

The film 54 is a compound designed to form a film that is sufficiently strong to adhere to the container until time of reconstitution. In some embodiments, the film 54 is an edible film, containing the concentrated content material and additives to provide the structure and adhesion properties; the additives are ideally odorless and tasteless so as to not interfere with the taste and scent of the content material. The concentration level of the content may determine the amount, strength and combination of components used to provide the adhesive property of the film.

The film 54 is solvent-soluble so as to allow reconstitution into a liquid using the concentrated content. The film 54 may be a composite film composed of MC or HPMC and various kinds of solids, such as beeswax. In some embodiments, the composition of the film has a water vapor permeability comparable to low density polyethylene (LDPE). Such composite films may incorporate a polymer-lipid bilayer formed from aqueous ethanolic solutions of cellulose ether fatty acids.

The adhesive characteristics of the film 54 may comprise a gel, jelly, starch, gelatin, or other material, such as pullulan. The gel may be a hydrogel in a semi-solid, jelly-like state. Various starches may be incorporated to provide adhesion to the concentrated content and/or adhesive property to maintain the bond between the film 54 and the cup 52. A starch may be an amylose film, amylopectin film, or cassava-based material. Some embodiments employ a protein material, including a gelatin film, a silk (fibroin) film, or a casein film. Some embodiments incorporate a fiber thread polysaccharide series, including pullulan film, guar gum resolvent film, soybean polysaccharide film, or agar-agar film. Similarly, some embodiment incorporate a cellulose material, such as those described hereinabove, HPC film, HPMC film, or carboxymethyl cellulose film. In some embodiments, arabinoxylan film, alginate sodium film, callageenan film, pectin film may be used within or with the film 54.

Cassava starch provides a transparent and colorless flexible film and may be prepared with minimal to no chemical treatment. The functional properties of edible films are enabled and enhanced through the use of substances having starch properties, including chain conformation, molecular bonding, crystallinity, and water content.

The use of starch-based films provides the desirable properties to adhere to a cup or container, while not interfering with the properties of the concentrated content. For example, the presence of the hydroxyl group on carbon 6 of the cyclic part of glucose is directly correlated with the functional properties of cassava starch films.

A pullulan polymer also makes a film that is edible, approximately tasteless, and transparent. In one embodiment, the concentrated content layer is concentrated mouthwash containing pullulan, menthol, sucralose, potassium acesulfame, copper gluconate, polysorbate 80, chrondrus cripus (carrageenan), glyceryl oleate, thymol, eucalyptol, menthyl salicylate, ceratonia siliqua gum, propylene glycol, xanthan gum and starch material. The content is concentrated and then the starch material is added.

FIG. 7 illustrates a bottle according to some embodiments, wherein the bottle 62 includes a film 64 containing a concentrated content. The film 64 is positioned within the bottle, and reacts when a solvent is added to the bottle. The solvent may be water, or may be a cleaning product. In some embodiments, the solvent is water and the film 64 is reconstituted to a full strength content material when water is added.

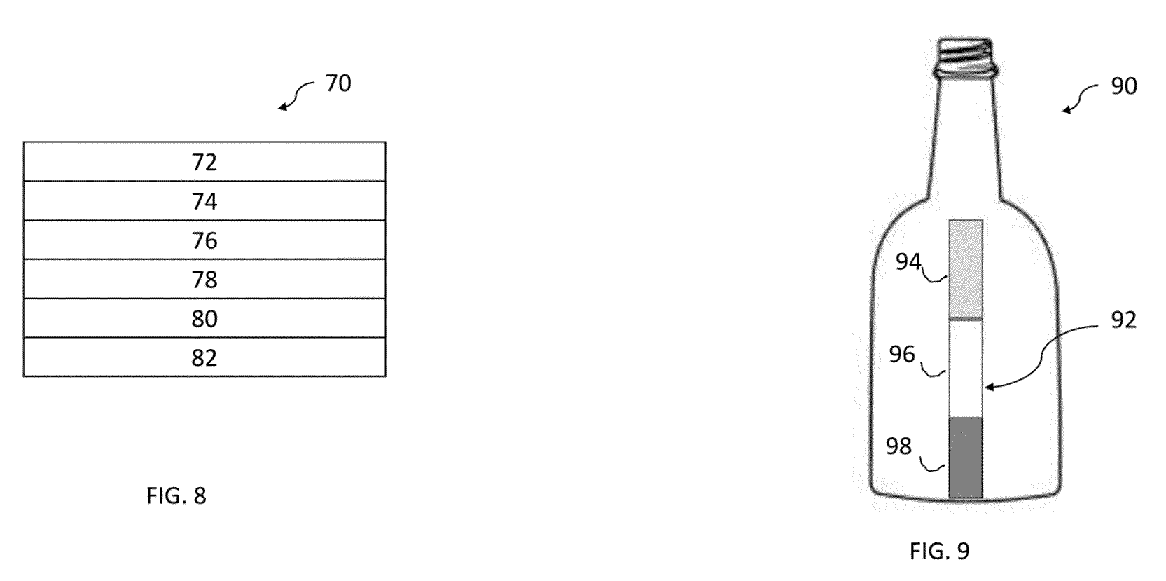

FIG. 8 illustrates a film structure 70 having a base layer 82 which includes an adhesive for adhering to a container, and an active content layer 80. In this embodiment, the film structure 70 is designed to enable multiple reconstitutions using a solvent. The layers of film structure 70 are alternated so as to isolate each content layer. A protective, sacrificial layer 78 is placed on the content layer 80, and so on. The layers 78 and 74 are applied to protect layers 76 and 80, respectively, from degradation. When the film 70 is applied to a container and a predetermined amount of solvent added to the container, the content film 72 reacts with the solvent to reconstitute the original material. After the material 72 is reconstituted, the film 70 does not degrade further due to protective layer 74. This layer may be a gel or other edible material that provides limited isolation from the solvent. The layer 74 dissolves over time in the presence of the solvent, enabling the reconstitution of the content layer 72. In this way, the container may be used multiple times to reconstitute the content. Any number of layers may be incorporated to enable multiple reconstitutions or use of the container.

FIG. 9 illustrates an embodiment wherein a container 90 has an inner surface on which a film 92 adheres. The film 92 has multiple portions, each having a different trigger color to indicate a condition of the material content of the film 92. The first portion 94 is a first color when the content material of the film 92 is concentrated and no solvent has been introduced. The first portion 94 turns to a second color, distinct from the first color, on addition of the solvent. The color indicates that some of the content material has been used. The second portion 94 of the film 90 acts in a similar way to indicate a concentration level of the content material. The third portion 96 makes the same change to indicate a condition of the content material. The three portions 92, 94, 96 provide indicators as to the current concentration of the content material.

In one embodiment the color of the strip is an indicator of the concentration strength of the content material or the active ingredient. In some embodiments, the color of the reconstituted liquid indicates the content strength or other parameter of the active ingredient.

In some applications, the color of the film changes to identify an incorrect solvent, such as where alcohol or vinegar is added to a content material that is to be reconstituted with water. In this case, a first color or condition of the film indicates a match between the content material and solvent, and a second color or condition of the film indicates a mis-match. This may be particularly applicable where the content material is a medicine or pharmaceutical product and the solvent is critical to proper activation of the medicine.

A variety of applications are considered, such as to provide a flavored drink concentrate in the film which is then deposited, sprayed or otherwise formed on the container surface, typically an interior surface or area or surface area over which the reconstituted content passes during use or consumption. The novel methods and apparatus presented herein may be used to reduce the transport weight of drinks, mouthwash, cleaners, and so forth.

As disclosed the present invention may be used in a variety of applications. By avoiding the need for a separate bottle of liquid, having its own container and requiring transport and storage by the user, the present invention reduces waste associated with the current methods. Further, there are a variety of methods to provide the film, such as to use a confection product or other edible substance to provide the adhesive. The present invention further provides a sanitary way to maintain oral hygiene. A variety of other applications may incorporate the novel concepts, such as where the film is a cleaning substance that reconstitutes with water or other solvent. Similarly, the film may be designed to indicate a strength of the content material of the film, strength of the reconstituted content material or other indicator related to the content material.

What is claimed:

1. A container, comprising:
an inner surface; and
a film adhering to the inner surface, the film comprising a concentrated content material and an adhesive material, wherein the concentrated content material is selected to react with a solvent introduced into the container to reconstitute an active ingredient of the content material.

2. The container as in claim 1, wherein the solvent is water, the content material is mouthwash liquid and the film reacts with the solvent to form the mouthwash liquid.

3. The container as in claim 2, wherein the film adheres to one of: a bottom portion or wall portion of the container.

4. The container as in claim 1, wherein the film comprises a starch material.

5. The container as in claim 1, wherein the film comprises a gel material.

6. The container of claim 1, wherein the film adheres to a top rim of the container, and the film reacts with the solvent as the solvent is poured out of the container.

7. The container of claim 6, wherein the film reacts with the solvent as a user drinks from the container.

8. An oral hygiene product, comprising:
a disposable cup; and
an oral hygiene film coupled to an inner portion of the disposable cup, wherein
the oral hygiene film reacts with water to form mouthwash.

9. The oral hygiene product of claim 8, wherein the oral hygiene film is deposited on an inner surface of the disposable cup.

10. The oral hygiene product of claim 8, wherein the oral hygiene film comprises a concentrated form of the mouthwash.

11. An edible film, comprising:
a content material that reacts with a solvent to transform into a liquid form of the content material; and
an adhesive material to adhere the content material to a surface.

12. The edible film of claim 11, wherein the adhesive material is a starch.

13. The edible film of claim 11, wherein the content material is concentrated, wherein addition of the solvent to the concentrated content material reconstitutes the content material.

14. The edible film of claim 8, further comprising:
an isolation layer overlaid on the content material and the adhesive material.

15. The edible film of claim 14, wherein the isolation layer comprising an adhesive material.

16. The edible film of claim 11, wherein the film indicates a strength of the content material.

17. The edible film of claim 11, wherein the content material includes an active ingredient, and wherein the film indicates a concentration of the active ingredient.
